(19) United States
(12) Patent Application Publication
(10) Pub. No.: US 2018/0188224 A1
(43) Pub. Date: Jul. 5, 2018

(54) METHOD FOR CONSTRUCTING A SENSORY SPACE

(71) Applicant: CENTRE SCIENTIFIQUE ET TECHNIQUE DU BATIMENT (CSTB), Champs Sur Mame (FR)

(72) Inventors: Fabrice DE OLIVEIRA, Nantes (FR); Philippe HUMEAU, Nantes (FR); Gwenaelle HAESE, La Chapelle Sur Erdre (FR); Pierre LE CLOIREC, Rennes (FR)

(21) Appl. No.: 15/741,565

(22) PCT Filed: Jul. 1, 2016
(86) PCT No.: PCT/FR2016/051672

§ 371 Date: Jan. 3, 2018

(30) Foreign Application Priority Data
Jul. 3, 2015 (FR) 15 56350

(51) Int. Cl. G01N 33/18; A61B 5/00

(52) U.S. Cl. CPC G01N 33/186; A61B 5/04847; A61B 5/7239; A61B 5/4017

(57) ABSTRACT

Disclosed is a method for constructing a sensory space of an individual, including: the individual performing a sensory evaluation with at least 4 basic stimuli representative of the sense to be studied, the stimuli having an intensity (or power or level) lower than or in the vicinity of the detection threshold of the sense to be studied; recording signals relating to at least one physiological indicator having reacted during the stimuli; extracting physiological parameters from the recorded signals of the at least one physiological indicator; identifying physiological parameters differentiating the physiological responses; constructing a sensory space based on the physiological responses (PRSS) from the identified differentiating physiological parameters by statistical treatment.

The present invention relates to a method for constructing a sensory space based on physiological responses (ESRP) (from discriminating physiological parameters) and also to a method for sensory analysis comprising the positioning of a sample within a reference sensory space (ESRP) making it possible to predict the chemical and/or sensory characteristics thereof.

Sensory analysis, also referred to as sensory metrology, is a scientific field used to evaluate, measure, analyze and interpret responses to products perceived by the sense of sight, smell, touch, taste and hearing. The main aim of sensory analysis is to translate the desires and preferences of consumers into well-defined and tangible properties of a given product. The hypothesis used to associate them is that part of the sensations is structured by preference. By comparing and analyzing the characteristics of the product that consumers like or do not like, sensory analysis contributes to identifying the positive and negative aspects and to adapting them to satisfy the tastes of consumers.

It is beneficial to study several aspects of a product: its quality, its intensity and its hedonic dimension. Quality is defined as a set of inherent characteristics such as the description of an image, of a smell, of a feel, of a taste, or of a sound. Intensity represents the degree of presence of a characteristic in the product. The hedonic dimension of a product is its ability to bring pleasure or displeasure. There are three main categories of sensory analysis, which aim to evaluate, respectively, quality, intensity or the hedonic dimension, and which use panelists chosen on the basis of specific criteria. For the evaluation of the hedonic dimension, some methods of sensory analysis may rely on a naive (untrained) panel, but require a large number of panelists to obtain representative results. On the other hand, conventional methods of sensory analysis enabling the evaluation of the quality and intensity rely on groups of panelists, whom it is generally necessary to teach and train, especially to reduce the subjectivity of each of the panelists. The reliability of the measurements, and particularly the reproducibility, is then highly dependent on the teaching and expertise of the panelists. Teaching and training of the panelists are all the more critical in certain fields in which the characteristics to be evaluated are close to detection thresholds. For example, the sensory analysis of drinking water remains extremely complicated due to the low concentrations of sapid and odorizing substances, and to the complexity in describing such a medium. In all cases, the necessity of teaching and training panelists and/or of a large number of panelists makes using sensory analyses costly in terms of time and money. Moreover, even when they are conducted by a trained panel, sensory analyses give results which are generally interpreted qualitatively.

Moreover, some methods of sensory analysis propose using physiological indicators. The emotional state associated with sensory stimulations is expressed for an individual through behavioral and neurovegetative reactions. These reactions or physiological effects of the activity of the autonomic nervous system (ANS) may be discerned more or less directly by physiological indicators (electrodermal response, heart rate, thermovascular indicators, analysis of pupil diameter, electroencephalogram, etc.). Several studies have demonstrated the existence of correlations between sensory stimulations and certain physiological indicators. These studies have especially attempted to correlate the physiological responses of individuals to their preferences or to different characteristics of a stimulus. The physiological parameters extracted from the monitoring of physiological indicators vary depending on the measurement (statistical parameters, shape parameters, spectral parameters, etc.). Studies using a multi-parameter approach have generally extracted statistical parameters of mean or of standard deviation, making it possible to qualitatively classify the physiological reactions or to directly analyze the distributions by statistical comparison tests on a panel of subjects. However, these methods do not enable a significant discrimination of the intensity and/or of the overall assessment of the stimuli.

Moreover, these studies have essentially focused on concentrations well above the detection thresholds (at least 10 times greater) and have generally compared stimuli with diametrically opposed hedonic valences.

In this context and to their credit, the inventors have developed a method for sensory analysis based on the construction of an individual sensory space, that is to say specific to an individual, from physiological measurements, making it possible to overcome the abovementioned limitations. In particular, the method for sensory analysis according to the invention is applicable to naive subjects and thus makes it possible to circumvent the problem of long, restrictive and costly training that conventional methods for sensory analysis involve. In addition, the individual approach of this method enables a reference sensory space to be obtained which is reliable and stable over time. The individual is likened to a sensor for the sensory characteristics to be studied, which makes it possible to overcome difficulties of verbalization and problems of subjectivity associated with perception. It is a question of constructing the sensory space which, in the same way as a calibration function, translates the measured individual physiological response to a stimulus into a sensory response or even into characteristic values of the product (chemical or physical). This is due to the particular predisposition of the body which means that an individual reacts personally to the influence of external agents. The method is therefore based on the principle of idiosyncrasy, that is to say on making use of the particular predisposition of an individual which means that an individual reacts personally to the influence of external agents. Thus, it is possible to obtain information relating to the quality, intensity and/or hedonic dimension of an unknown sample often in a more reliable manner than with a conventional method of analysis, especially for stimuli close to the perception thresholds.

Thus, the present invention relates to a method for constructing a sensory space of an individual and also to a method for the sensory analysis of a sample based on the use of this sensory space.

The method for constructing a sensory space specific to an individual according to the invention comprises:
conducting a sensory evaluation of at least 4 basic stimuli representative of the sense to be studied by the individual, the stimuli having an intensity (or strength or level) lower than or close to the detection threshold of the sense to be studied;
recording signals relating to at least one physiological indicator, during the sensory evaluation, which reacted during the stimuli;
extracting physiological parameters from the recorded signals of said at least one physiological indicator;
identifying discriminating physiological parameters from the physiological responses;
constructing a sensory space from the discriminating physiological parameters identified by statistical treatment.

The method for constructing an individual sensory space is based on the coming together of physiological responses and sensory responses measured simultaneously while conducting a sensory evaluation of basic stimuli representative of the sense to be studied by the individual, the stimuli having an intensity (or strength or level) lower than or close to the detection threshold. The sensory responses are obtained by self-assessment by the individual, using a sensory questionnaire. The physiological responses are obtained by recording the signals relating to at least one physiological indicator.

Conducting a sensory evaluation of at least 4 basic stimuli representative of the sense to be studied thus consists in determining at least 4 stimuli associated with the sense in question and with a family of compounds for the sense studied, for example flavors for taste.

Thus, mention may be made, for taste, of stimuli which correspond to basic flavors, namely sour, bitter, salty, sweet and umami, but also of secondary tastes: medicinal, plant-like, stale, metallic, earthy, musty, chemical, spicy, fruity, floral and aromatic. These stimuli are capable of only stimulating the sense of taste and of simulating positive and negative hedonic valences.

For smell, the following stimuli may be chosen, comprising the following types of odors: septic, sulfurous-marshy, chemical (chlorine), hydrocarbon, medicinal, plant-like, fruity, floral, earthy, stale, rancid and phenolic.

The method may also be used to study other senses such as hearing and touch.

For each of the stimuli, several parameters will be studied: its quality, its intensity (or strength or level), and its hedonic dimension. The quality is defined as “qualitas”, that is to say a set of inherent characteristics. Intensity (or strength or level) represents the degree of presence of a characteristic in the product. Intensity corresponds to stimuli of taste or smell, strength to stimuli of touch and level to stimuli of hearing. The hedonic dimension of a product is its ability to bring pleasure or displeasure.

These parameters are determined by conventional sensory analysis by means of a questionnaire defined to this end. Each tester is placed in a climate-controlled neutral environment suitable for tasting, as defined by standard ISO 8589 (2010). The stimuli to which they are each subjected are transmitted in a random order; a “blank” stimulus is used at the start of the analysis. The number of stimuli at each sensory analysis session is limited so as to avoid any sensory fatigue. For example, in the case of taste stimuli, a tasting session will be limited to approximately 10 samples to be tested. In order to also determine the detection thresholds of the different stimuli, for a given stimulus, different concentrations or strengths or levels are tested. In the present invention, “stimuli having an intensity (or strength or level) lower than or close to the detection threshold of the sense to be studied” is understood to mean stimuli which are at a concentration (or strength or level) lower than the concentration (or strength or level) detectable by the sense to be studied, and which may extend up to a concentration (or strength or level) lower than 3, preferably lower than 2 times the concentration (or strength or level) detectable by the sense to be studied.

Indeed, a physiological response to a stimulus may be demonstrated even when the individual does not perceive this stimulus, that is to say that, even below the detection threshold of the sense to be studied, a physiological response will be demonstrated and will enable the invention to be carried out.

By way of example, the detection thresholds for the four primary tastes, sour, sweet, salty and bitter, are given by the concentrations of the following representative compounds:
“sour”: citric acid: detection threshold: 0.384-0.5 g·l⁻¹,
“bitter”: caffeine: detection threshold: 0.05-0.35 g·l⁻¹,
“salty”: NaCl: detection threshold: 0.584 g·l⁻¹,
sweet: sucrose: detection threshold: 6.846 g·l⁻¹.

The physiological indicators which make it possible to establish the relationship between the quality, intensity and hedonic value of the stimuli are indicators of the central nervous system and/or of the autonomic nervous system, measured synchronously. Of course, several physiological indicators may be used cumulatively.

Physiological indicators of the central nervous system which may be used are the activity of the central nervous system which may be measured by electroencephalography (EEG) or by functional near-infrared spectroscopy.

Physiological indicators of the autonomic nervous system which may be used are heart rate, electrodermal response (EDR), skin temperature, skin microcirculation, gustofacial reflex measured by electromyography, pupil response, respiratory response, and blood pressure.

For each of the physiological indicators in question, different parameters were measured and those which appeared as the most discriminating for the physiological responses associated with the stimuli and in association with the intensity and/or the hedonic valence and/or the physical or chemical characteristics (for example concentration of the sample) of the stimuli were retained.

According to an embodiment of the invention, when several physiological indicators are measured, the measurements are carried out in a synchronized manner, that is to say according to the same timescale and at the same acquisition frequency, for example 1 kHz. For each indicator, different parameters are measured.

By way of example, if the physiological indicator in question is heart rate (HR), it will be measured from the electrocardiogram (ECG) and following the RR time detection (detection of the peak R of the QRS complex of the ECG, construction of a tachogram). The ECG measurement may be carried out by a 3-lead ECG differential amplifier or equivalent, the electrodes being positioned according to the recommendations for cardiac measurements and the following different parameters (use of algorithms developed under Matlab®) may be measured in a synchronized manner:
Duration: Total duration of the reaction
Duration at maximum: Duration before the maximum HR is reached
Maximum bpm: Maximum heart rate reached, in bpm
Increase in bpm: Increase in the HR in bpm compared to the 15 s preceding the stimulation
% increase: % increase in the HR in bpm compared to the 15 s preceding the stimulation
Standard deviation, band 0 Hz-0.15 Hz: Square root of the sum of the energy present in the frequency band 0-0.15 Hz over the total duration of the reaction
Standard deviation, band 0.15 Hz-0.4 Hz: Square root of the sum of the energy present in the frequency band 0.15-0.4 Hz over the total duration of the reaction
Standard deviation, band 0.4 Hz-0.5 Hz: Square root of the sum of the energy present in the frequency band 0.4-0.5 Hz over the total duration of the reaction
(VLF+LF)/HF: Standard deviation 0 Hz-0.15 Hz band/standard deviation 0.15-0.4 Hz band.

By way of example, if the physiological indicator in question is EDR, it may be measured by means of a GSR FE116 (Galvanic Skin Response) amplifier, and the parameters which will be extracted may be:
AGradientMax Ordinate at GradientMax
AGradientMin Ordinate at GradientMin
APeak Ordinate at peak, not relative to baseline
Height Value of Apeak minus baseline
GradientMax At each sampling point in the zone between the Start and the End, a gradient is calculated by five-point linear regression centered on the sampling point. The maximum of these gradients is taken for GradientMax.
GradientMin As for GradientMax, the minimum of these slopes is taken for GradientMin (where the minimum is the steepest descending gradient).
AreaPeak Area of the zone between the Start and the End
GradientDescent Gradient of the straight line between the points located at the crossing point of 90% and 10% of the Height of the peak between the peak and the End
GradientRise Gradient of the straight line between the points located at the crossing point of 10% and 90% of the Height of the peak between the Start and the Peak
TDescent Duration between the points located at the crossing point of 90% and 10% of the Height of the peak between the Start and the peak
DurationPeak Duration between the Start and APeak
TGradientMax Duration between the Start and GradientMax
TGradientMin Duration between the Start and GradientMin
TRise Duration between the points located at the crossing point of 10% and 90% of the Height of the peak between the Start and the peak
Width30, Width50, WidthX (where X is equal either to 30, 50 or 90) is calculated as
Width90 the duration at X % of the height of the peak starting from the line between the rise and the descent
Width Duration between the Start and the End.

Also by way of example, if the physiological indicator is the skin microcirculation, it may be measured by means of a Periflux PF 5010 (Perimed AB, Sweden) apparatus and the simultaneously measured parameters which will be extracted may be similar to the parameters extracted on the EDR signals.

The identification of the discriminating parameters is carried out by statistical analysis. The different methods of statistical analysis which may be used are especially analysis of variance (ANOVA) which makes it possible to evaluate the effect of one or more qualitative factors on a quantitative variable for which the measurements have been carried out with several repetitions (Pages, J., 2010, Statistique générale pour utilisateurs. Méthodologie. [General statistics for users. Methodology] PUR, Rennes); analysis of correlations by virtue of the Pearson (or Spearman) correlation coefficient, which is suitable for continuous variables and is also the most widely used (Labbe, D., Rytz, A. & Hugi, A., 2004, Training is a critical step to obtain reliable product profiles in a real food industry context. Food Quality and Preference, 15, 341-348), for measuring the performance of the panel and also for measuring the link between the physiological variables and the sensory variables; principal component analysis (ACP) which is applied to datasets which cross individuals in rows and quantitative variables in columns, and the aim of which is to summarize the variability contained in all of the data by a minimum of uncorrelated dimensions referred to as principal components (Escofier, B. & Pagès, J., 2008, Analyses factorielles simples et multiples: objectifs, méthodes et interprétation. [Single- and multi-factorial analyses: aims, methods and interpretation.] Dunod, Paris); factorial correspondence analysis (FCA), which is applied to contingency tables crossing the modalities of two qualitative variables and the aim of which is to obtain a classification of the rows and a classification of the columns, and then to link them to one another (Escofier, B. & Pagès, J., 2008, Analyses factorielles simples et multiples: objectifs, méthodes et interprétation. Dunod, Paris). Other methods of multivariate statistical analysis or exploratory data analysis techniques may also be used (Linear discriminant analysis: LDA, Partial least squares: PLS, neural networks, etc.).

Once the discriminating parameters have been separately determined for each of the individuals, an individual “products” sensory space is constructed.

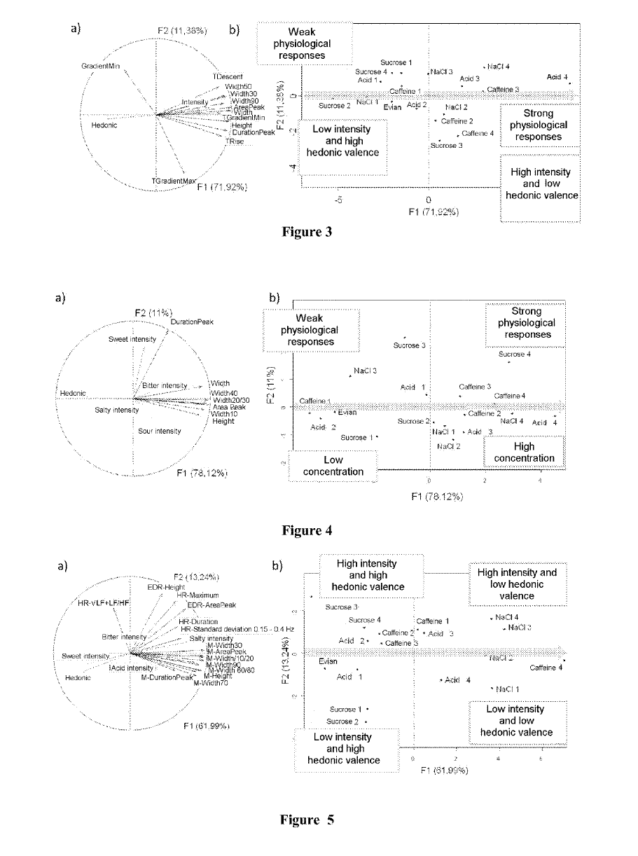

By way of example, a sensory space is represented in FIG. 3b.

This products sensory space, unique to the individual, corresponds to the “calibration” of the individual doing the testing and may subsequently be used for the systematic evaluation of samples by this individual.

Thus, according to another aspect, the invention also relates to a method for the sensory analysis of a sample, comprising:
constructing a sensory space of an individual as defined above;
evaluation of the sample by the individual, associated with recording the signals relating to at least one physiological indicator corresponding to the discriminating physiological parameters used for constructing the sensory space;
extracting the discriminating physiological parameters of the sample;
positioning the sample in the sensory space using the discriminating physiological parameters of the sample.

By virtue of this method of sensory analysis, stimuli close to the threshold values for detection by conventional, purely sensory methods, are detected. The sensitivity of the method is greater than that of conventional methods, even with testers who have not undergone specific training but who have specific discriminating physiological parameters.

Thus, this method may make it possible to evaluate unknown samples to measure where they will be located compared to known stimuli, that is to say to determine their quality, intensity and hedonic value, especially.

The invention also relates to the use of the method of sensory (sensory-physiological) analysis for analyzing products, such as drinking water, requiring very precise sensory analyses which must be carried out by high-quality testers who are subject to intense prior training.

The invention will now be described in more detail by means of examples given solely by way of illustration and the appended drawings, in which:

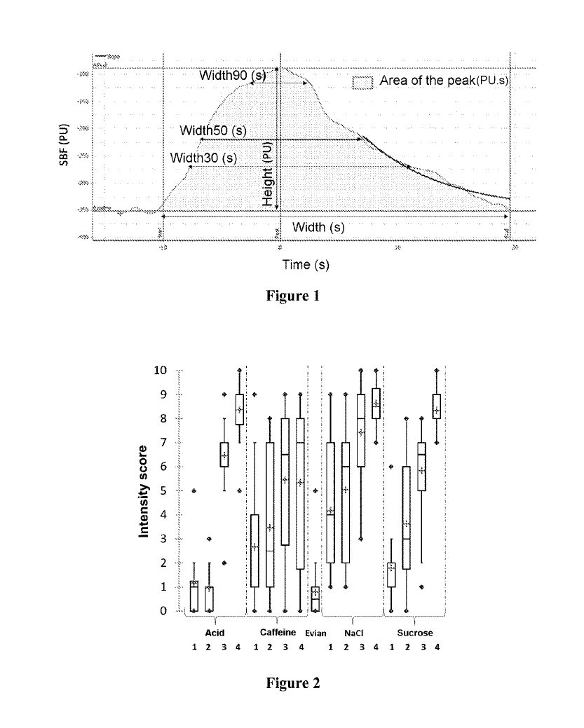

FIG. 1 is a graph of the analysis of the peak induced by a taste stimulus, produced by the LabChart® software.

FIG. 2 represents the boxes for the dispersion of the intensity scores obtained with each taste stimulus and for each concentration (1, 2, 3, 4, cf. table 1), averaged over 4 subjects with 6 repetitions each (i.e. a total of 24 intensity scores per product) and 12 repetitions for the Evian sample (i.e. a total of 48 intensity scores).

FIG. 3a) is the representation of the variables (active in solid line and illustrative in dotted line); and FIG. 3b) is the representation of the taste stimuli obtained by PCA (F1-F2) on the blood microcirculation data obtained for one individual.

FIG. 4a) is the representation of the variables (active in solid line and illustrative in dotted line); and FIG. 4b) is the representation of the taste stimuli obtained by PCA (F1-F2) on the electrodermal response data obtained for one individual (subject 1).

FIG. 5a) is the representation of the variables (active in solid line and illustrative in dotted line); and FIG. 5b) is the representation of the taste stimuli obtained by PCA (F1-F2), obtained for one individual on data originating from several physiological indicators: skin microcirculation, electrodermal response and heart rate.

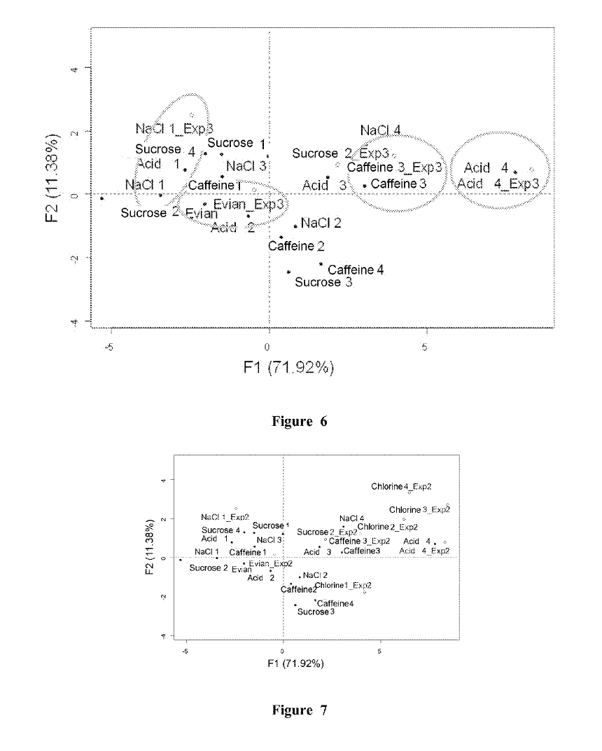

FIG. 6 presents an example of use of the ESRP (constructed from microcirculation) from the projection of test product having reference concentrations.

FIG. 7 presents an example of use of the ESRP (constructed from microcirculation) from the projection of new product having a different sensory modality (chlorine odor).

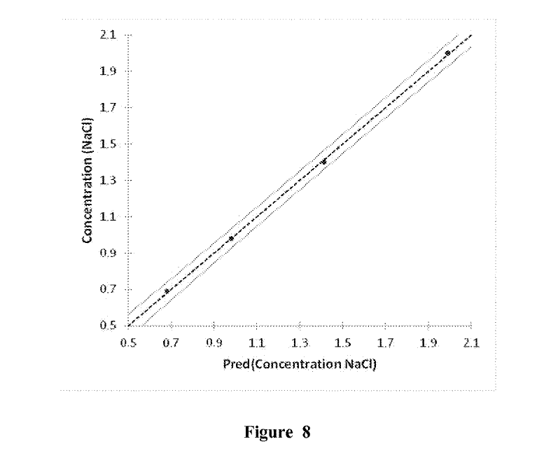

FIG. 8 illustrates the quality of the prediction models for the salty flavor resulting from the ESRP parameters by comparing the real concentrations of the reference samples and their predicted concentrations (Q²=0.995).

### EXAMPLES

### Example 1: Construction of a Sensory Space Intended to be Used to Test the Quality of Tap Water on a Panel of Experts

The aim of this example is to construct a sensory space ESRP based on measurements of skin microcirculation, in order to use it as reference to characterize problems of odor and of taste in water samples. The sensory dimensions studied are the hedonic valence and the intensity of the product.

### Material and Methods

### Subjects

In this test, four non-smoking volunteers (2 men and 2 women) were chosen from 12 people for their performance in taste recognition. Their mean age was 32 years, ranging from 24 to 40 years. They confirmed they were not following any medical treatment and did not have any olfactory or gustatory disorders and were informed of the tests they were to be subjected to. These four people were trained for several months to recognize and detect the four basic tastes, and also regarding the procedures of sensory and physiological analysis.

### Taste Stimuli

The four basic tastes (sweet, salty, sour and bitter) were used. The solutions were prepared with citric acid for the sour taste, caffeine for the bitter taste, sodium chloride for the salty taste and sucrose for the sweet taste (all purchased from Sigma Aldrich, France) in Evian water. Evian water was also used as control and blank. Each taste was prepared with the four concentrations mentioned in table 1.

TABLE 1
Concentrations (mmol · l⁻¹) for each taste stimulus^{a}.

| | Citric acid | Caffeine | NaCl | Sucrose |
|---|---|---|---|---|
| Dilution | (sour) | (bitter) | (salty) | (sweet) |
| 1 | 1.6 | 0.72 | 12 | 8 |
| 2 | 2.0 | 0.88 | 17 | 13 |
| 3 | 2.5 | 1.13 | 24 | 21 |
| 4 | 3.1 | 1.39 | 34 | 35 |

^{a}For example, in this table the dilution 1 of the citric acid (corresponding to a concentration of 1.6 mmol · l⁻¹ of citric acid) will be referred to as Acid 1 in this example; the same naming will be used for the other concentrations.

Evian mineral water was chosen for its neutral taste (Teillet, E., Schlich, P., Urbano, C., Cordelle, S. & Guichard, E., 2010, Sensory methodologies and the taste of water. Food Quality and Preference, 21, 967-976) and because it has been widely used in studies of physiological reactions in response to taste stimuli (Rousmans, S., Robin, O., Dittmar, A. & Vernet-Maury, E., 2000 Autonomic nervous system responses associated with primary tastes. Chemical Senses, 25, 709-718; Robin, O., Rousmans, S., Dittmar, A. & Vernet-Maury, E., 2003, Gender influence on emotional responses to primary tastes. Physiology & Behavior, 78, 385-393; Leterme, A., Brun, L., Dittmar, A. & Robin, O. (2008) Autonomic nervous system responses to sweet taste: Evidence for habituation rather than pleasure. Physiology & Behavior, 93, 994-999). The detection of the threshold concentrations of the basic tastes are relatively high:
approximately 2 mmol·l⁻¹ for citric acid,
approximately 10 mmol·l⁻¹ for NaCl
approximately 20 mmol·l⁻¹ for sucrose (Purves, D., Augustine, G.-J., Fitzpatrick, D. & Hall, W.-C., 2005, Neurosciences. De Boeck Supérieur, Brussels) and
between 0.26 et 1.80 mmol·l⁻¹ for caffeine (Robinson, K. M., Klein, B. P. & Lee, S. Y., 2005, Utilizing the R-index measure for threshold testing in model caffeine solutions. Food Quality and Preference, 16, 283-289).

The concentrations of the solutions were chosen from these threshold values and from prior standardized taste tests (AFNOR, 2012) in order to have concentrations close to the detection thresholds.

The amount of solution to be tested was standardized at 10 ml. In order to impregnate the whole of the oral cavity, the subjects kept the solution in their mouth for 5 s before swallowing it.

### System for Recording Skin Microcirculation

A Periflux PF 5010 (Perimed AB, Sweden) system was chosen, which enables non-invasive monitoring of blood perfusion in the capillaries, arterioles and venules.

The unit of blood perfusion is arbitrary and corresponds to the product of the mean rate of displacement of the red blood cells by their concentration. The system Periflux PF 5010 has a 780 nm, 1 mm diameter laser beam without thermal effect. An optical fiber probe PR407 is used. The low-energy laser beam is transmitted by the probe to the tissue. A portion of the light reflects off the static structures and another portion of the light reflects off the moving blood cells. When the light reflects off a moving cell, the wavelength is modified, which corresponds to the Doppler effect. The scattered light collected by the optical fiber is used to calculate the perfusion value. The measurement is expressed in perfusion units (PU). Recording was carried out on the pad of the index finger of the non-dominant hand, due to the high level of vascularization of this zone. The perfusion signals were recorded by an 8-channel PowerLab PL35088/35 acquisition system; the data were collected at a frequency of 1 kHz and analyzed by the LabChart® software (AD instruments Ltd, United Kingdom).

The resultant signal was first inverted (multiplied by −1) such that the analysis of the peak by the LabChart® software was possible, and its value was multiplied by 100 to convert it from volts into PU. The signal was then filtered by a low-pass filter having a cut-off frequency of 0.6 Hz to remove the characteristic frequency of the heart beat which was not relevant in the present analysis. Then, the peak was analyzed manually by selecting the whole of the peak and analyzing it with the LabChart® software (FIG. 1).

Several shape parameters were extracted from the signal (i.e. amplitude, duration, gradient). They are given in table 2.

TABLE 2
List of the parameters extracted by the LabChart ® software and their calculation.

| Parameters | Calculation |
|---|---|
| AGradientMax | Ordinate at GradientMax |
| AGradientMin | Ordinate at GradientMin |
| APeak | Ordinate at peak, not relative to baseline |
| Height | Value of Apeak minus baseline |
| GradientMax | At each sampling point in the zone between the Start and the End, a |
| | gradient is calculated by five-point linear regression centered on the |
| | sampling point. The maximum of these gradients is taken for GradientMax. |
| GradientMin | As for GradientMax, the minimum of these slopes is taken for GradientMin |
| | (where the minimum is the steepest descending gradient). |
| AreaPeak | Area of the zone between the Start and the End |
| GradientDescent | Gradient of the straight line between the points located at the crossing point |
| | of 90% and 10% of the Height of the peak between the peak and the End |
| GradientRise | Gradient of the straight line between the points located at the crossing point |
| | of 10% and 90% of the Height of the peak between the Start and the Peak |
| TDescent | Duration between the points located at the crossing point of 90% and 10% |
| | of the Height of the peak between the Start and the peak |
| DurationPeak | Duration between the Start and APeak |
| TGradientMax | Duration between the Start and GradientMax |
| TGradientMin | Duration between the Start and GradientMin |
| TRise | Duration between the points located at the crossing point of 10% and 90% |
| | of the Height of the peak between the Start and the peak |
| Width30, | WidthX (where X is equal either to 30, 50 or 90) is calculated as the |
| Width50, | duration at X % of the height of the peak starting from the line between the |
| Width90 | rise and the descent. |
| Width | Duration between the Start and the End |

### Procedure

The sessions were individual and the subjects were asked not to eat or drink anything for at least 1 h before the test. They arrived 15 min before the start of the session in order to acclimatize to the environmental conditions, in particular the temperature of the room (kept constant at 23° C.) and to be resting seated on a comfortable chair. The 16 solutions for testing and two additional samples of water containing solely Evian water, added to the references, were presented monadically following a test order determined from a “balanced incomplete block design”. During a session, 9 samples out of 18 were presented to minimize sensory fatigue. In addition, a sample of Evian was always tested as a “blank” sample in order to avoid the first-order effect (Lawless, H. T. & Heymann, H., 2010, Sensory evaluation of food: Principles and practices. Springer, New York) and was excluded from the analysis. A light signal was sent to give the instruction to test the sample. When the disturbance to the nervous system returned to its baseline level (after approximately 1 min), a second light signal indicated to the subjects that they should complete a questionnaire regarding the stimulus tested previously. The first part of the questionnaire was linked to the identification of the taste (i.e. determining the quality of the taste); the subjects had to choose between five labels: sweet, salty, sour, bitter or neutral. They then had to assign a taste intensity score on an 11-point scale (from 0 “not intense” (i.e. taste of Evian only) to 10 “very intense”) and finally to give a hedonic score on an 11-point scale (from 0 “very unpleasant” to 10 “very pleasant”, 5 being “neutral”).

During this second phase, the subjects were able to rinse their mouth with Evian water, and awaited the following light signal which indicated that they could test the following stimulus. This procedure was repeated for each of the 9 stimuli. A session lasted approximately 45 min including a debriefing at the end of the test to ensure understanding of the sense of the signal variations. The experiment was repeated 12 times per subject, meaning that the data are composed of 6 data sets per subject and per product and 12 data sets for the Evian product per subject. Thus, the database consists of 432 samples.

### Statistical Analysis

All the analyses were carried out with SensoMineR software (Husson, F. & Lê, S. (2009) SensoMineR: Sensory data analysis with R. R package version 1.10. http://CRAN.R-project.org/package=SensoMineR) and FactoMineR software (Lê, S., Josse, J. & Husson, F. (2008) FactoMineR: An R Package for Multivariate Analysis. Journal of Statistical Software, 25, 1-18) produced in R (version 2.14.1) and XLSTAT-Pro 2010.3.01.

### Sensory Responses

The effect of the taste on the hedonic and intensity scores was analyzed by an analysis of variance (ANOVA) including the product effect, the subject effect and the product-subject interaction. The subject effect was fixed because there were only 4 subjects and the results could only be applied to a particular schema. The differences were considered to be significant at a level of 0.05.

### Variations in the Skin Microcirculation

Principal Component Analysis (PCA) was used:
to study the individuals (i.e. the taste stimuli): two stimuli are close if they share similar results, the point of interest being the variability between the individuals,
to study the variables (i.e. the physiological parameters): it enables visualization of the correlations between the physiological variables, in order to find synthetic variables,
to link the two studies by characterizing the groups of individuals with variables (Escofier, B. & Pagès, J., 2008, Analyses factorielles simples et multiples: objectifs, méthodes et interprétation. Dunod, Paris).

The SKBF responses were analyzed individually by one-way ANOVA (product effect), the subjects having their own “preferential channel” (Lacey, J. I., Bateman, D. E. & VanLEHN, R., 1953, Autonomic response specificity: An experimental study. Psychosomatic Medicine, 15, 8-21). Indeed, some subjects responded to SKBF variations, others with EDR variations, and physiological measurements are known to show significant individual differences, which lead to the necessity to individualize the analysis (Johannes, B. & Gaillard, A. W. K., 2014, A methodology to compensate for individual differences in psychophysiological assessment. Biological psychology, 96, 77-85).

Finally, correlations between the mean hedonic and intensity scores per product and the mean responses of the nervous system were calculated by means of the Pearson coefficient. The differences were considered to be significant at a level of 0.05.

### Results

### Sensory Evaluation

### Level of Recognition

The salty and sweet tastes were correctly identified by the panel for all the concentrations, with a level of recognition of 100% for the 3 highest scores (table 3). The highest sour concentrations were also perfectly recognized and the bitter taste was relatively well identified (92% and 96%, respectively, of correct responses for the concentrations 3 and 4). However, low sour concentrations were not recognized and were most commonly classified as “neutral” (54% and 38%, respectively, for the concentrations 1 and 2). The Evian mineral water was often perceived as bitter (in 38% of the cases), but as it was used for all the dilutions, the results are comparable.

TABLE 3
Individual recognition scores for each taste stimulus and the levels of recognition (%) for the four subjects.

| | | Citric acid | | | | Caffeine | | | | | NaCl | | | | Sucrose | | | |
|---|---|---|---|---|---|---|---|---|---|---|---|---|---|---|---|---|---|---|
| | | 1 | 2 | 3 | 4 | 1 | 2 | 3 | 4 | Evian | 1 | 2 | 3 | 4 | 1 | 2 | 3 | 4 |
| Subject 1 | Sour | 1 | 1 | 6 | 6 | 0 | 0 | 0 | 0 | 1 | 0 | 0 | 0 | 0 | 0 | 0 | 0 | 0 |
| | Bitter | 1 | 1 | 0 | 0 | 5 | 6 | 6 | 6 | 4 | 0 | 0 | 0 | 0 | 0 | 0 | 0 | 0 |
| | Neutral | 3 | 4 | 0 | 0 | 1 | 0 | 0 | 0 | 2 | 0 | 0 | 0 | 0 | 0 | 0 | 0 | 0 |
| | Salty | 0 | 0 | 0 | 0 | 0 | 0 | 0 | 0 | 1 | 6 | 6 | 6 | 6 | 0 | 0 | 0 | 0 |
| | Sweet | 1 | 0 | 0 | 0 | 0 | 0 | 0 | 0 | 4 | 0 | 0 | 0 | 0 | 6 | 6 | 6 | 6 |
| Subject 2 | Sour | 0 | 2 | 6 | 6 | 0 | 0 | 0 | 0 | 0 | 0 | 0 | 0 | 0 | 0 | 0 | 0 | 0 |
| | Bitter | 0 | 0 | 0 | 0 | 6 | 4 | 6 | 6 | 3 | 0 | 0 | 0 | 0 | 0 | 0 | 0 | 0 |
| | Neutral | 5 | 3 | 0 | 0 | 0 | 1 | 0 | 0 | 7 | 0 | 0 | 0 | 0 | 0 | 0 | 0 | 0 |
| | Salty | 0 | 0 | 0 | 0 | 0 | 0 | 0 | 0 | 0 | 6 | 6 | 6 | 6 | 0 | 0 | 0 | 0 |
| | Sweet | 1 | 1 | 0 | 0 | 0 | 1 | 0 | 0 | 2 | 0 | 0 | 0 | 0 | 6 | 6 | 6 | 6 |
| Subject 3 | Sour | 1 | 1 | 6 | 6 | 0 | 1 | 0 | 0 | 1 | 0 | 0 | 0 | 0 | 0 | 0 | 0 | 0 |
| | Bitter | 2 | 4 | 0 | 0 | 6 | 5 | 6 | 6 | 3 | 0 | 0 | 0 | 0 | 0 | 0 | 0 | 0 |
| | Neutral | 3 | 1 | 0 | 0 | 0 | 0 | 0 | 0 | 8 | 0 | 0 | 0 | 0 | 3 | 0 | 0 | 0 |
| | Salty | 0 | 0 | 0 | 0 | 0 | 0 | 0 | 0 | 0 | 6 | 6 | 6 | 6 | 0 | 0 | 0 | 0 |
| | Sweet | 0 | 0 | 0 | 0 | 0 | 0 | 0 | 0 | 0 | 0 | 0 | 0 | 0 | 3 | 6 | 6 | 6 |
| Subject 4 | Sour | 1 | 4 | 6 | 6 | 0 | 0 | 0 | 1 | 0 | 0 | 0 | 0 | 0 | 0 | 0 | 0 | 0 |
| | Bitter | 3 | 1 | 0 | 0 | 3 | 5 | 4 | 5 | 8 | 1 | 0 | 0 | 0 | 0 | 0 | 0 | 0 |
| | Neutral | 2 | 1 | 0 | 0 | 3 | 1 | 2 | 0 | 4 | 0 | 0 | 0 | 0 | 0 | 0 | 0 | 0 |
| | Salty | 0 | 0 | 0 | 0 | 0 | 0 | 0 | 0 | 0 | 5 | 6 | 6 | 6 | 0 | 0 | 0 | 0 |
| | Sweet | 0 | 0 | 0 | 0 | 0 | 0 | 0 | 0 | 0 | 0 | 0 | 0 | 0 | 6 | 6 | 6 | 6 |
| Level of | Sour | 13 | 33 | 100 | 100 | 0 | 4 | 0 | 4 | 4 | 0 | 0 | 0 | 0 | 0 | 0 | 0 | 0 |
| Recognition % | Bitter | 25 | 25 | 0 | 0 | 83 | 84 | 92 | 96 | 38 | 4 | 0 | 0 | 0 | 0 | 0 | 0 | 0 |
| | Neutral | 54 | 38 | 0 | 0 | 17 | 8 | 8 | 0 | 44 | 0 | 0 | 0 | 0 | 12 | 0 | 0 | 0 |
| | Salty | 0 | 0 | 0 | 0 | 0 | 0 | 0 | 0 | 2 | 96 | 100 | 100 | 100 | 0 | 0 | 0 | 0 |
| | Sweet | 8 | 4 | 0 | 0 | 0 | 4 | 0 | 0 | 12 | 0 | 0 | 0 | 0 | 88 | 100 | 100 | 100 |

### Intensity Scores

The intensity scores were strongly linked to the level of recognition (r=0.75, p<0.001). They confirmed that the low acid concentrations 1 and 2 were scored as weakly intense like the Evian mineral water (respectively 1.2±1.5, 1.0±1.0 and 0.8±1.2); they were just below the detection thresholds. The product effect was very significant for the medium intensity scores (p<0.001). The concentrations 4 of the sour, salty and sweet tastes had the highest scores, respectively 8.4±1.2, 8.6±1.0 and 8.3±0.9. The bitter solutions were less differentiated from one another and gave the highest standard deviations; the concentrations 3 and 4 were not significantly different. The concentrations 1 and 2 of the salty taste were also not significantly different. The only taste for which each concentration was scored significantly differently from the previous one was sweet (FIG. 2).

The product-subject interaction was significant (p<0.001); the effect of the product was not the same depending on the subject. This interaction was mainly due to the subject having had difficulties in discriminating between the bitter tastes. Training tends to reduce this effect in the context of a description of the characteristics of the products (Lawless and Heymann, 2010).

### Hedonic Scores

The hedonic scores were significantly different between the products (p<0.001). The Evian mineral water obtained a neutral hedonic score (5.2±1.5), as did the weakest concentrations of the different tastes, close to the detection thresholds. As the concentration increased, the hedonic scores of the sour, bitter and salty solutions decreased while the hedonic scores for the sweet solutions increased in line with their concentrations.

The acid concentration 4, the caffeine concentrations 3 and 4 and each of the NaCl concentrations had hedonic scores significantly lower than the other solutions (respectively 3.3±2.4, 3.5±1.8, 3.5±2.3 and 1.3±0.9), while all the sweet solutions obtained significantly higher hedonic scores (7.0±2.6 for the highest sucrose concentration).

The product-subject interaction was significant (p<0.001); the effect of the product was not the same depending on the subject. In the case of hedonic scores, this interaction was not problematic, since several subjects were able, for example, to appreciate the sour taste while other subjects found it very unpleasant.

These sensory results confirmed the choice of the basic tastes and the choice of the different concentrations. Indeed, the 17 stimuli were distributed on the scale of intensity, from the detection thresholds to the highest intensities, and enabled the experiment over the whole of the hedonic scale, even if the hedonic valence is taken as an individual item of information in this example.

The physiological evaluation is focused on the results from subject 3 only.

### Physiological Evaluation, ESRP: Skin Blood Microcirculation

PCA was carried out in order to visualize the data with the physiological parameters extracted from the individual variations in SKBF from subject 3 (cf. FIG. 1 and table 2). This analysis enables the representation of all the variables on a correlation circle (FIG. 3a) and all the products in a two-dimensional space (FIG. 3b). The first chart represented 83.3% of the initial variability.

The first PCA axis was characterized by a group of variables of duration and of amplitude of physiological response (FIG. 4a). In this way, the acid 3 and 4, caffeine 3 and 4 and NaCl 4 products located on the right-hand portion of the chart (FIG. 4b) tended to have high values for the variables “width”, “height” and “peak area”.

The second axis separated the “gradient” values. The products located in the top portion of the graph tended to cause a more rapid drop in the signal after the stimulation, while the products in the bottom portion induced a slower return to the baseline level before stimulation.

The hedonic and intensity variables were treated as illustrative variables; they were not involved when constructing the axes. However, they appear to be highly correlated to the physiological variables. The perceived intensity was greatly and positively correlated with the first dimension (r=0.72, p=0.001), while the hedonic variable was greatly and negatively correlated with the first dimension (r=−0.65, p=0.005). The products perceived as intense and unpleasant appeared to be characterized by stronger physiological responses, unlike the pleasant and intense stimuli.

According to ANOVA, the most discriminating variables for the products were the signal width, i.e. the duration thereof (p<0.001), and the area of the peak (p<0.001). Acid 4, NaCl 4 and caffeine 3 induced a significantly stronger disturbance than the others. Since the parameters are mathematically linked, the height of the peak, i.e. the amplitude thereof, was also a discriminating parameter (p=0.004) for the acid 4 and caffeine 3 stimuli.

Correlation analysis confirmed these results. The width of the signal was significantly correlated to the intensity (r=0.68, p=0.003) and to the pleasant character (r=−0.63, p=0.007), and also the peak area (r=0.65, p=0.004 and r=−0.65, p=0.005 respectively).

### Example 2: Construction of a Sensory Space Intended to be Used to Test the Quality of Tap Water on a Naive Panel

The aim of this example is to construct a sensory space ESRP based on measurements of skin microcirculation, electrodermal response and heart rate, in order to use it as reference to characterize problems of odor and of taste in water samples. The sensory dimensions studied are the hedonic valence and the intensities of the product as a function of the 4 basic tastes (sweet intensity scale, salty intensity scale, bitter intensity scale and sour intensity scale).

### Subjects

The selection of the panelists for choosing the subjects participating in the final phase of the experiments was conducted in the same way as that presented during the preliminary experiments phase. The criteria for inclusion remained the same. The volunteers were recruited according to the following criteria:
being between 18 and 45 years old,
being a non-smoker,
not following any particular medical treatment other than oral contraception,
not having any problems with taste or smell.

Thirty one subjects, nineteen women and twelve men (aged from 18 to 40 years) participated in the selection tests, split over two sessions, after having been informed of the experimental conditions and having signed a consent form.

The instructions to follow in order to minimize the impact of undesirable parameters on sensitivity to the tastes of the water were the following:
do not drink coffee or tea in the hour before the tasting,
avoid the use of perfume, aftershave or lipstick on the day of the tasting,
do not eat any sweets, chewing gum or throat lozenges before the tasting,
do not eat spicy food or drink alcohol before the tasting,
brush your teeth as far from the time of the tasting as possible.

### Taste Stimuli

The four basic tastes (sweet, salty, sour and bitter) were used. The solutions were prepared with citric acid for the sour taste, caffeine for the bitter taste, sodium chloride for the salty taste and sucrose for the sweet taste (all purchased from Sigma Aldrich, France) in Evian water. Evian water was also used as control and blank. Each taste was prepared with the four concentrations mentioned in table 4.

TABLE 4
Concentrations used for each flavor (in g · l⁻¹)

| | Sour | Bitter | Salty | Sweet |
|---|---|---|---|---|
| Dilution | (citric acid) | (caffeine) | (NaCl) | (sucrose) |
| 1 | 0.38 | 0.14 | 0.48 | 2.59 |
| 2 | 0.41 | 0.17 | 0.69 | 4.32 |
| 3 | 0.48 | 0.22 | 0.98 | 7.20 |
| 4 | 0.60 | 0.27 | 1.40 | 12.00 |

### Physiological Evaluation, ESPR: Electrodermal Response

The ESPR is presented in FIGS. 4a) and b) constructed from relevant parameters extracted from the electrodermal response. These figures respectively present the correlation circle for the factorial plane F1-F2 and the factorial plane F1-F2 with the cloud of individuals. All the variables are positively correlated with the first axis; the variable “DurationPeak” is also positively correlated with the second axis. On the cloud of individuals, all the products at the highest concentration 4 are located to the right of the plane (cumulative contributions: 39.63%). These products assume higher values for all the physiological parameters. Conversely, the products at low concentrations, caffeine 1, acid 2 and the sample of Evian water, are located to the right of the plane (cumulative contributions: 43.48%) and have lower values for all the parameters. The first factor therefore opposes the low intensities to the high intensities. The axis 2 is supported by the products sucrose 3 and 4, which contribute to more than 58% of its inertia. These two products take higher values for the “DurationPeak” variable.

As shown in the preliminary study, the link between the sensory variables from self-assessment and the physiological responses may be visualized by virtue of the projection of the former into illustrative variables on the PCA. The correlation is then calculated between the means obtained per product.

The correlation circle and the first factorial plane are represented in FIG. 4.

The first factor of the PCA (F1) is also negatively correlated to the hedonic scores (r=−0.506, p<0.05). Examining the hedonic scores given by subject 1, this correlation is explained by the fact that the hedonic scores for the products decrease relative to their increasing concentration. However, the levels of the hedonic scores are not the same between the sweet stimuli and the bitter stimuli, for example. Indeed, subject 1 appreciates the sweet stimuli to a degree, although the score is lower for the strong stimulus than for the weak, while said subject does not really appreciate the bitter stimuli. The EDR in subject 1 is therefore more linked to the intensity of the stimuli than to their hedonic dimension.

The link between the physiological reactions and the real concentration of the tastes in the water was studied by virtue of correlation coefficients. The correlations were calculated per taste.

The representation of the ESRP space obtained, FIG. 4, is directly linked to the concentrations. Indeed, the first factor of the PCA is significantly positively correlated with the concentration of citric acid and with the concentration of sucrose (r=0.829, p<0.20 and r=0.878, p<0.20, respectively) and is positively correlated with the concentration of NaCl and of caffeine, without reaching significance (r=0.301 and r=0.792, respectively). The second factor is also positively correlated with the concentration of caffeine and the concentration of sucrose (r=0.690 and r=0.756, respectively).

The study of the correlations between each discriminating physiological parameter and the concentration shows a significant positive link between the height and the area of the peak and the concentration of citric acid (r=0.895, p<0.20 and r=0.908, p<0.10, respectively). Regarding the concentrations of caffeine and of NaCl, they are positively and significantly correlated with the time taken to return to a baseline physiological level. The concentration of sucrose is positively correlated with the duration at the peak and with the duration of the disturbance (r=0.937, p<0.10 and r=0.955, p<0.05, respectively).

### Physiological Evaluation, ESPR: Skin Blood Microcirculation, Electrodermal Response, Heart Rate

This example relates a sensory space to an individual who responded on several channels. This example is presented in FIGS. 5 a) and b). The results obtained for EDR and for heart rate variations for this individual are now presented. Only two EDR parameters are discriminating for the products: the area (p<0.20) and the height of the peak (p<0.01) and four for HRV: the maximum in bpm (p<0.10), the duration and standard deviation of the band 0.15-0.4 H (p<0.20), and the VLF+LF/HF (p<0.05). These six variables were added to the previous PCA with the discriminating microcirculation variables. The area of the peak for EDR is significantly correlated with the hedonic valence of the stimuli (r=−0.365, p<0.20), and also the duration, the maximum and the standard deviation of the band 0.15-0.4 Hz of the heart rate variations (respectively r=−0.819, p<0.0001, r=−0.601, p<0.05 and r=−0.659, p<0.01). The maximum reached in terms of heart rate is significantly positively linked to the salty and bitter intensities declared, and the duration and the standard deviation of the band 0.15-0.4 Hz are also positively correlated to the salty intensity. It is therefore the salty stimuli which have the most impact on heart variations. The two EDR variables are directly linked to the concentration of NaCl and of sucrose: the area of the peak is significantly and positively correlated (respectively r=0.935, p<0.10 and r=0.860, p<0.20), as is the height (respectively r=0.971, p<0.05 and r=0.854, p<0.20).

### Example 3: Use of the ESRPs with Reference Products

The ESRP may be used as a response space, onto which products may be projected in order to be compared to standard products (products originally used to construct the sensory space). This projection is carried out from the analysis of the physiological parameters (selected for the construction of the ESRP) linked to the physiological response of these new products.

FIG. 6 illustrates the projection of the new measurements having reference concentrations. The projection of these new measurements, as illustrative individuals (indexed as _Exp3), shows that the position of the different samples remains relatively unchanged on the first factor and that the classification of the samples is the same, with the exception of sucrose 2. Indeed, the coordinates of the sample of Evian water, of NaCl 1, of caffeine 3 and of acid 4 for the first experiment are respectively −2,0, −3,4, 3,0 and 7,8 on the first factor of the PCA and are respectively −0,5, −2,4, 3,9 and 8,3 during the second experiment. These results show that, even one year later, the measurements are stable and reproducible over time. The representation of the individual ESPR is therefore robust and may be effectively reused for subsequent measurements. It now remains to be studied whether it is possible to use it for new samples to be evaluated, with other tastes or odors than those used for its construction.

### Example 4: Use of the ESRPs with New Products (Chlorine Odor)

The odor of chlorine seems relevant to examine, for several reasons. Indeed, one of the most problematic odors in French drinking water is that of chlorine. Chlorine is more an odor than a taste, since it is detected in water based on an olfactory component and not on a gustatory component at the concentrations supplied through the distribution networks. The odor of chlorine added to Evian water was therefore used to apply the method to the concrete problem of drinking water in France.

### Subject

As a reminder, the ESPR used here corresponds to a subject having skin microcirculation as preferential channel. The representation of the reference ESPR obtained by virtue of the subject's physiological responses is therefore already known.

### Olfactory Stimuli

The concentrations were therefore chosen as a function of the results obtained on French subjects. Four different concentrations were used (two at the level of the thresholds and two more obvious) and are described in table 5.

TABLE 5
Concentrations used for the chlorine-like flavor (in mg Cl₂ · l⁻¹)

| | NaClO |
|---|---|
| Dilution | (Chlorine) |
| 1 | 0.10 |
| 2 | 0.17 |
| 3 | 0.32 |
| 4 | 1.00 |

Since chlorine is a highly volatile product, the stock solution was prepared a few minutes before the start of the session and the solutions were diluted in the tasting glasses once the subject was prepared and set up in the tasting booth.

### Materials and Method

Only the relevant physiological indicator is measured, that is to say the microcirculation. The parameters to be extracted and the standardization of the data are already known, which leads to a gain in time at the point of processing the information.

### Results

The measurements declared are presented in table 6.

TABLE 6
Chlorine intensity and hedonic scores declared

| | Chlorine-like | |
|---|---|---|
| | intensity | Hedonic score |
| Chlorine 1 | 3.50 | 4.50 |
| Chlorine 2 | 4.25 | 3.25 |
| Chlorine 3 | 4.50 | 3.00 |
| Chlorine 4 | 4.25 | 3.50 |

Projection of the New Samples onto the ESPR: Electrodermal Response
FIG. 7 presents the projection of the values of the physiological parameters measured for the chlorine samples. The chlorine samples are all located in the right-hand portion of the factorial plane. Given their coordinates on the first factor, it appears that all the concentrations of chlorine, even the weakest, induced strong physiological reactions. The study of the correlations between the two first factors of the PCA and the characteristics of the chlorine-like samples confirm this interpretation. Indeed, the correlation between the hedonic score of the chlorine-like stimuli and the two first factors is significant and negative (respectively r=−0.922, p<0.10 and r=−0.889, p<0.20). There are positive correlations between the intensity declared, the concentration of chlorine and the two first factors, but they do not reach the significance threshold of 20%. The correlation is positive between the first axis and the intensity declared (r=0.777, p=0.22) and between the second factor and the concentration (r=0.662, p=0.34). It appears that the second axis takes on a greater importance for the chlorine-like samples and that there is an ordered relation according to F1 and F2. These results show that the chlorine odor induces much stronger responses than the basic tastes.

### Example 5: Use of the ESRPs to Predict the Concentrations of a Product

Models were constructed for the flavors sour, bitter and salty, in order to obtain the prediction of the chemical characteristics of the stimuli in water. The aim is to demonstrate a direct link between the physiological reactions and the chemical composition of the water samples. The quality of each model obtained is presented in table 7.

TABLE 7
Quality of the models for predicting the concentration of
samples by type of taste; partial least squares model

| Type of taste | Q² cum |
|---|---|
| Sour | 0.980 |
| Bitter | 0.920 |
| Salty | 0.995 |

FIG. 8 illustrates the quality of the prediction models for the salty flavor by comparing the real concentrations of the reference samples and their predicted concentrations.
These different models were used to predict the reference solution concentrations. The following table presents the real and predicted concentrations associated with the PLS models based on the physiological parameters alone. It is noted that there is a good match between the different concentrations.

TABLE 8
Comparisons of the real and predicted concentrations
according to each of the models resulting from the
PLS regression on the samples by taste type

Concentration (in g · l−1)

| | real | predicted |
|---|---|---|
| Acid 4_Exp2 | 0.60 | 0.65 ± 0.01 |
| Caffeine 3_Exp2 | 0.22 | 0.25 ± 0.03 |
| NaCl 1_Exp2 | 0.69 | 0.87 ± 0.02 |

1. A method for constructing a sensory space specific to an individual, comprising:
conducting a sensory evaluation of at least 4 basic stimuli representative of the sense to be studied by the individual, the stimuli having an intensity (or strength or level) lower than or close to the detection threshold of the sense to be studied;
recording signals relating to at least one physiological indicator which reacted during the stimuli;
extracting physiological parameters from the signals recorded of said at least one physiological indicator;
identifying discriminating physiological parameters from the physiological responses;
constructing a sensory space based on the physiological responses (ESRP) from discriminating physiological parameters identified by statistical treatment.

2. The method as claimed in claim 1, wherein, for taste, the 4 stimuli will be sour, bitter, salty and sweet flavors.

3. The method as claimed in claim 1, wherein the physiological indicator(s) is/are chosen from indicators of the central nervous system and/or the autonomic nervous system.

4. The method as claimed in claim 3, wherein the physiological indicator(s) of the central nervous system is (are) the activity of the central nervous system, which may be measured by electroencephalography (EEG) or by functional near-infrared spectroscopy.

5. The method as claimed in claim 3, wherein the physiological indicator(s) of the autonomic nervous system is (are) chosen from heart rate, electrodermal response, skin microcirculation, gustofacial reflex measured by electromyography, pupil response, respiratory response, skin temperature and blood pressure.

6. The method as claimed in claim 1, wherein the discriminating physiological factors are identified by a statistical analysis chosen from analysis of variance (ANOVA), Pearson coefficient, principal component analysis (PCA), factorial correspondence analysis (FCA), or combinations of these different methods.

7. A method for the sensory analysis of a sample, comprising:
constructing a sensory space of an individual as defined in claim 1;
evaluation of the sample by the individual;
recording the signals relating to at least one physiological indicator corresponding to the discriminating physiological parameters used for constructing the sensory space;
extracting the discriminating physiological parameters of the sample;
positioning the sample in the sensory space using the discriminating physiological parameters of the sample.

8. The method of analysis as claimed in claim 7, wherein the sample to be analyzed is a sample of drinking water.

9. The method as claimed in claim 2, wherein the physiological indicator(s) is/are chosen from indicators of the central nervous system and/or the autonomic nervous system.

10. The method as claimed in claim 2, wherein the discriminating physiological factors are identified by a statistical analysis chosen from analysis of variance (ANOVA), Pearson coefficient, principal component analysis (PCA), factorial correspondence analysis (FCA), or combinations of these different methods.

11. The method as claimed in claim 3, wherein the discriminating physiological factors are identified by a statistical analysis chosen from analysis of variance (ANOVA), Pearson coefficient, principal component analysis (PCA), factorial correspondence analysis (FCA), or combinations of these different methods.

12. The method as claimed in claim 4, wherein the discriminating physiological factors are identified by a statistical analysis chosen from analysis of variance (ANOVA), Pearson coefficient, principal component analysis (PCA), factorial correspondence analysis (FCA), or combinations of these different methods.

13. The method as claimed in claim 5, wherein the discriminating physiological factors are identified by a statistical analysis chosen from analysis of variance (ANOVA), Pearson coefficient, principal component analysis (PCA), factorial correspondence analysis (FCA), or combinations of these different methods.

14. The method as claimed in claim 9, wherein the discriminating physiological factors are identified by a statistical analysis chosen from analysis of variance (ANOVA), Pearson coefficient, principal component analysis (PCA), factorial correspondence analysis (FCA), or combinations of these different methods.

15. A method for the sensory analysis of a sample, comprising:
constructing a sensory space of an individual as defined in claim 2;
evaluation of the sample by the individual;
recording the signals relating to at least one physiological indicator corresponding to the discriminating physiological parameters used for constructing the sensory space;
extracting the discriminating physiological parameters of the sample;
positioning the sample in the sensory space using the discriminating physiological parameters of the sample.

16. A method for the sensory analysis of a sample, comprising:
constructing a sensory space of an individual as defined in claim 3;
evaluation of the sample by the individual;
recording the signals relating to at least one physiological indicator corresponding to the discriminating physiological parameters used for constructing the sensory space;
extracting the discriminating physiological parameters of the sample;
positioning the sample in the sensory space using the discriminating physiological parameters of the sample.

17. A method for the sensory analysis of a sample, comprising:
constructing a sensory space of an individual as defined in claim 4;
evaluation of the sample by the individual;
recording the signals relating to at least one physiological indicator corresponding to the discriminating physiological parameters used for constructing the sensory space;
extracting the discriminating physiological parameters of the sample;
positioning the sample in the sensory space using the discriminating physiological parameters of the sample.

18. A method for the sensory analysis of a sample, comprising:
constructing a sensory space of an individual as defined in claim 5;
evaluation of the sample by the individual;
recording the signals relating to at least one physiological indicator corresponding to the discriminating physiological parameters used for constructing the sensory space;
extracting the discriminating physiological parameters of the sample;
positioning the sample in the sensory space using the discriminating physiological parameters of the sample.

19. A method for the sensory analysis of a sample, comprising:
constructing a sensory space of an individual as defined in claim 6;
evaluation of the sample by the individual;
recording the signals relating to at least one physiological indicator corresponding to the discriminating physiological parameters used for constructing the sensory space;
extracting the discriminating physiological parameters of the sample;
positioning the sample in the sensory space using the discriminating physiological parameters of the sample.

20. A method for the sensory analysis of a sample, comprising:
constructing a sensory space of an individual as defined in claim 9;
evaluation of the sample by the individual;
recording the signals relating to at least one physiological indicator corresponding to the discriminating physiological parameters used for constructing the sensory space;
extracting the discriminating physiological parameters of the sample;
positioning the sample in the sensory space using the discriminating physiological parameters of the sample.
